(19) United States
(12) Patent Application Publication
(10) Pub. No.: US 2008/0035478 A1
(43) Pub. Date: Feb. 14, 2008

(54) METHODS FOR FABRICATING A BIOSENSOR WITH A SURFACE TEXTURE

(76) Inventors: Greta Wegner, St. Anthony, MN (US); Natasha Popovich, Pompano Beach, FL (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/837,630

(22) Filed: Aug. 13, 2007

Related U.S. Application Data
(60) Provisional application No. 60/836,935, filed on Aug. 11, 2006.

(51) Int. Cl. C12M 1/00; B05D 5/12

(52) U.S. Cl. 204/403.01; 427/58

(57) ABSTRACT

The present invention includes a biosensor having a generally planar substrate including a surface texture configured to increase a surface area of the generally planar substrate. The biosensor may also include at least one conductive component at least partially formed on a portion of the surface texture. The invention also includes a method for manufacturing a biosensor. The method may include forming a surface texture on a generally planar substrate whereby the surface texture increases a surface area of the generally planar substrate. The method also includes at least partially forming at least one conductive component on a portion of the surface texture. The present invention also includes a reel having a generally planar substrate including a surface texture configured to increase a surface area of the generally planar substrate. The reel also includes at least one conductive component at least partially formed on a portion of the surface texture, and a plurality of registration points formed on the generally planar substrate.

This application claims priority to U.S. Provisional Patent Application No. 60/836,935 filed on Aug. 11, 2006, the contents of which are incorporated herein by reference.

### DESCRIPTION

1. Technical Field

The present invention relates to the field of diagnostic testing systems using electronic meters and, more particularly, a biosensor with a surface texture.

2. Background

Electronic testing systems are commonly used to measure or identify one or more analytes in a sample. Such testing systems can be used to evaluate medical samples for diagnostic purposes and to test various non-medical samples. For example, medical diagnostic meters can provide information regarding the presence, amount, or concentration of various analytes in human or animal body fluids In addition, diagnostic test meters can be used to monitor analytes or chemical parameters in non-medical samples such as water, soil, sewage, sand, air, beverage and food products or any other suitable sample.

Diagnostic testing systems typically include both test media, such as diagnostic test strips, and a test meter configured for use with the test media. Suitable test media may include a combination of electrical, chemical, and/or optical components configured to provide a response indicative of t he presence or concentration of an analyte to be measured. For example, some glucose test strips include electrochemical components, such as glucose specific enzymes, buffers, and one or more electrodes. The glucose specific enzymes may react with glucose in a sample, thereby producing an electrical signal that can be measured with the one or more electrodes. The test meter can then convert the electrical signal into a glucose test result.

There is a demand for improved test media. For example, in the blood glucose testing market, consumers consistently insist on test media that require smaller sample sizes, thereby minimizing the amount of blood needed for frequent testing. Consumers also demand robust performance and accurate results, and will not tolerate erroneous tests due to inadequate sample size. In addition, in all diagnostic testing markets, consumers prefer faster, cheaper, more durable, and more reliable testing systems.

Current methods of manufacturing diagnostic test media have inherent limits. For example, current methods for producing test media electrodes and depositing enzymes or other chemicals may have limited spatial resolution and/or production speeds. Furthermore, some production processes cannot be used to deposit some enzymes, chemicals, and electrodes. In addition, some production processes may be used to produce or deposit some test media components, such as electrodes or enzymes, while being incompatible with other components. Therefore, some test media production processes may require multiple production techniques, thereby increasing production cost and time, and decreasing product throughput.

There exists the need to mass-produce biosensors cost effectively and with high precision. The prior art references have several limitations solved by the current invention. For example, increasing the surface area of an electrode may increase the sensitivity of signal detection. Further, an increased surface area may enhance adhesion between a conductive layer and base layer and/or a reagent layer and a conductive layer. Other manufacturing methods may be used to lower the cost and/or increase the quality of electrode formation and biosensor performance.

Accordingly, there is a need for improved methods of manufacturing diagnostic testing systems.

### SUMMARY

A first aspect of the present invention includes a biosensor having a generally planar substrate including a surface texture configured to increase a surface area of the generally planar substrate. The biosensor also includes at least one conductive component at least partially formed on a portion of the surface texture.

A second aspect of the present invention includes a method for manufacturing a biosensor. The method includes forming a surface texture on a generally planar substrate whereby the surface texture increases a surface area of the generally planar substrate. The method also includes at least partially forming at least one conductive component on a portion of the surface texture.

A third aspect of the present invention includes a reel having a generally planar substrate including a surface texture configured to increase a surface area of the generally planar substrate. The reel also includes at least one conductive component at least partially formed on a portion of the surface texture, and a plurality of registration points formed on the generally planar substrate.

Additional aspects and advantages of the invention will be set forth in part in the description which follows, and in part will be apparent from the description, or can be learned by practice of the invention. The advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1A illustrates test media that can be produced using the methods of the present disclosure.

FIG. 1B illustrates a test meter that can be used with test media produced according to the methods of the present disclosure.

FIG. 1C illustrates a test meter that can be used with test media produced according to the methods of the present disclosure.

FIG. 2A is a top plan view of a test strip according to an exemplary embodiment of the invention.

FIG. 2B is a cross-sectional view of the test strip of FIG. 2A, taken along line 2B-2B.

FIG. 3A is a top view of a reel according to an exemplary disclosed embodiment of the invention.

FIG. 3B is an enlarged tip view of a feature set on the reel of FIG. 3A.

FIG. 4 is a view of a surface texture disposed on a base layer, according to an exemplary disclosed embodiment.

FIG. 5 is a scanning electron microscope image showing a plurality of conductive components formed on a base layer including a surface texture, according to an exemplary disclosed embodiment.

FIG. 6 is a top view of a conductive base layer of a test strip according to an exemplary embodiment of the invention.

FIG. 7 is a top view of a dielectric layer of a test strip according to an exemplary embodiment of the invention.

### DESCRIPTION OF THE EMBODIMENTS

Reference will now be made in detail to the exemplary embodiments of the invention, examples of which are illustrated in the accompanying drawings. Wherever possible, the same reference numbers will be used throughout the drawings to refer to the same or like parts.

In accordance with an exemplary embodiment, a biosensor manufacturing method is described. Many industries have a commercial need to monitor the concentration of particular constituents in a fluid. The oil refining industry, wineries, and the dairy industry are examples of industries where fluid testing is routine. In the health care field, people such as diabetics, for example, need to monitor various constituents within their bodily fluids using biosensors. A number of systems are available that allow people to test a body fluid (e.g. blood, urine, or saliva), to conveniently monitor the level of a particular fluid constituent, such as, for example, cholesterol, proteins or glucose.

A biosensor may include a test strip, which can be disposable, that may facilitate the detection of a particular constituent of a body fluid. The test strip can include a proximal end, a distal end, and at least one electrode. The proximal end of the test strip may include a sample chamber for receiving a body fluid to be tested. The sample chamber can be dimensioned and configured to draw a fluid sample into the sample chamber via capillary action. Electrodes positioned within the sample chamber may contact the fluid sample. The distal end of the test strip may be configured to operatively connect the test strip to a meter that may determine the concentration of the body fluid constituent. For example, the distal end of the test strip may include a plurality of electrical contacts configured to provide electrical connections between the electrodes within the sample chamber and the meter. The ends of the test strip may also include a visual and/or tactile distinguishable section, such as, for example, a taper, in order to make it easier for the user to operatively connect the test strip to the meter or apply a body fluid to the sample chamber.

Electrodes positioned within the sample chamber may include a working electrode, a counter electrode, and a fill-detect electrode. A reagent layer can be disposed in the sample chamber and may cover at least a portion of the working electrode, which can also be disposed at least partially in the sample chamber. The reagent layer can include, for example, an enzyme, such as glucose oxidase or glucose dehydrogenase, and a mediator, such as potassium ferricyanide or ruthenium hexamine, to facilitate the detection of glucose in blood. It is contemplated that other reagents and/or other mediators can be used to facilitate detection of glucose and other constituents in blood and other body fluids. The reagent layer can also include other components, such as buffering materials (e.g., potassium phosphate), polymeric binders (e.g., hydroxypropyl-methyl-cellulose, sodium alginate, microcrystalline cellulose, polyethylene oxide, hydroxyethylcellulose, and/or polyvinyl alcohol), and surfactants (e.g., Triton X-100 or Surfynol 485).

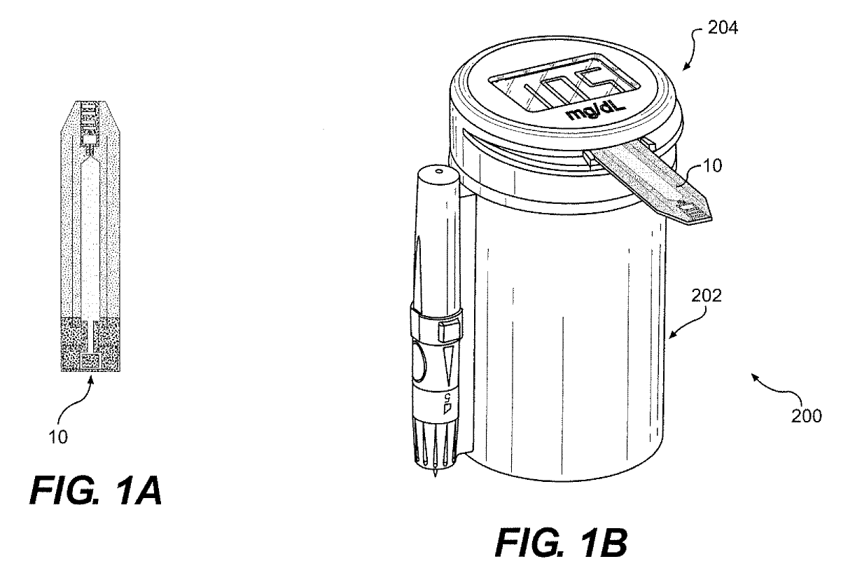

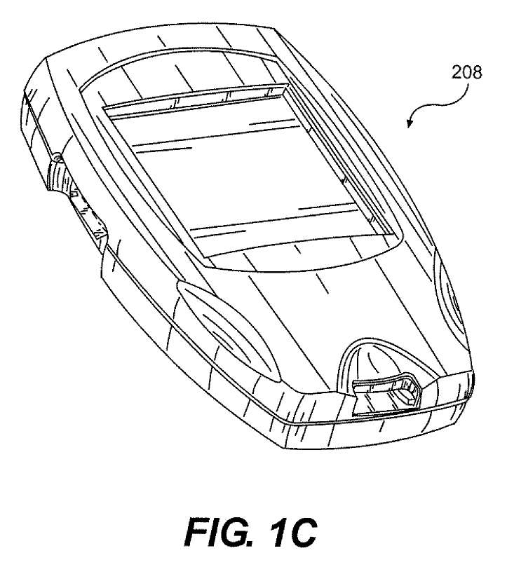

The present disclosure provides a method for producing a diagnostic test strip 10, as shown in FIG. 1A. Test strip 10 of the present disclosure may be used with a suitable test meter 200, 208, as shown in FIGS. 1B and 1C, to detect or measure the concentration of one or more analytes. The analytes to be tested for may include a variety of different substances, which may be found in biological samples, such as blood, urine, tear drops, semen, feces, gastric fluid, sweat, cerebrospinal fluid, saliva, vaginal fluids (including suspected amniotic fluid), culture media, and/or any other biologic sample. The one or more analytes may also include substances found in environmental samples such as soil, food products, ground water, pool water, and/or any other suitable sample.

As shown in FIG. 1A, test strip 10 are planar and elongated in design. However, test strip 10 may be provided in any suitable form including, for example, ribbons, tubes, tabs, discs, or any other suitable form. Furthermore, test strip 10 can be configured for use with a variety of suitable testing modalities, including electrochemical tests, photochemical tests, electro-chemiluminescent tests, and/or any other suitable testing modality.

Test meter 200, 208 may be selected from a variety of suitable test meter types. For example, as shown in FIG. 1B, test meter 200 includes a vial 202 configured to store one or more test strips 10. The operative components of test meter 200 may be contained in a meter cap 204. Meter cap 204 may contain electrical meter components, can be packaged with test meter 200, and can be configured to close and/or seal vial 202. Alternatively, a test meter 208 can include a monitor unit separated from storage vial, as shown in FIG. 1C. Any suitable test meter may be selected to provide a diagnostic test using test strip 10 produced according to the disclosed methods.

### Test Strip Configuration

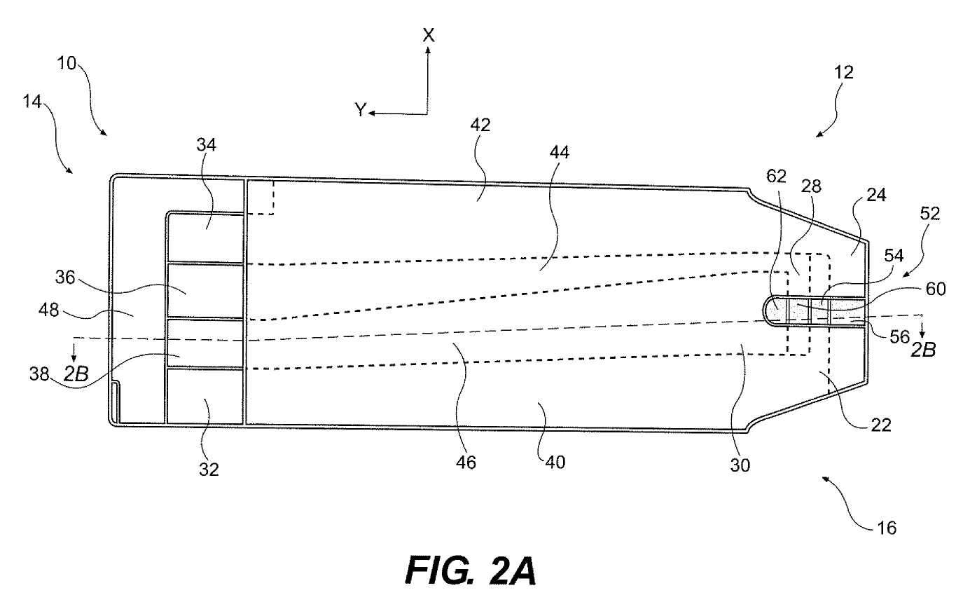

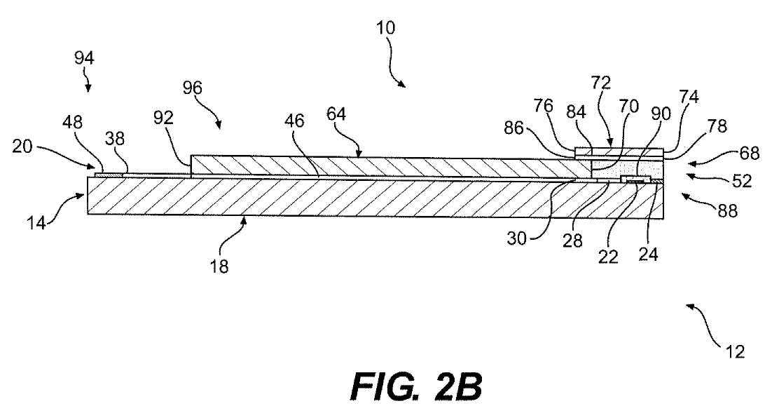

With reference to the drawings, FIGS. 2A and 2B show a test strip 10, in accordance with an exemplary embodiment of the present invention. Test strip 10 can take the form of a substantially flat strip that extends from a proximal end 12 to a distal end 14. In one embodiment, the proximal end 12 of test strip 10 can be narrower than distal end 14 to provide facile visual recognition of distal end 14. For example, test strip 10 may include a tapered section 16, in which the full width of test strip 10 tapers down to proximal end 12, making proximal end 12 narrower than distal end 14. If, for example, a blood sample is applied to an opening in proximal end 12 of test strip 10, providing tapered section 16 and making proximal end 12 narrower than distal end 14 can assist the user in locating the opening where the blood sample is to be applied. Alternatively, the distal end may be tapered. Further, other visual means, such as indicia, notches, contours or the like can be used.

Test strip 10 is depicted in FIGS. 2A and 2B as including a plurality of electrodes 22, 24, 28, 30. Each electrode may extend substantially along the length of test strip 10 to provide an electrical contact near distal end 14 of test strip 10 and a conductive region electrically connecting the region of the electrode near proximal end 12 to the electrical contact. In the exemplary embodiment of FIGS. 2A and 2B, the plurality of electrodes includes a working electrode 22, a counter electrode 24, a fill-detect anode 28, and a fill-detect cathode 30 at a proximal end 12 of test strip 10. Correspondingly, the electrical contacts can include a working electrode contact 32, a counter electrode contact 34, a fill-detect anode contact 36, and a fill-detect cathode contact 38 positioned at distal end 14 of test strip 10. The conductive regions may include a working electrode conductive region 40 that electrically connects the proximal end of working electrode 22 to working electrode contact 32, a counter electrode conductive region 42 that electrically connects the proximal end of counter electrode 24 to counter electrode contact 34, a fill-detect anode conductive region 44 that electrically connects the proximal end of fill-detect anode 28 to fill-detect contact 36, and a fill-detect cathode conductive region 46 that electrically connects the proximal end of fill-detect cathode 30 to fill-detect cathode contact 38.

In one embodiment, at least one electrode is partially housed within a sample chamber to allow contact with a fluid to be tested. For example, FIGS. 2B depicts test strip 10 as including a slot 52 that forms a portion of sample chamber 88 at proximal end 12. Slot 52 can define an exposed portion 54 of working electrode 22, an exposed portion 56 of counter electrode 24, an exposed portion 60 of fill-detect anode 28, and an exposed portion 62 of fill-detect cathode 30. Further, the exemplary embodiment includes an auto-on conductor 48 disposed near distal end 14 of strip 10 to allow the meter to determine that a test strip is operatively connected to the meter.

As shown in FIG. 2B, test strip 10 may have a layered construction. Test strip 10 includes a base layer 18 that may substantially extend along the entire length or define the length of test strip 10. Base layer 18 can be formed from an electrically insulating material and can have a thickness sufficient to provide structural support to test strip 10. For example, base layer 18 may be at least partially composed of any suitable polymer, such as, an acrylic, polyethylene terephthalate (PET), or plastic.

According to the exemplary embodiment of FIG. 2B, one or more conductive components 20 may be disposed on at least a portion of base layer 18. Conductive components 20 may include one or more electrically conductive elements, such as, for example, a plurality of electrodes. Conductive components 20 may include any suitable conductive or semi-conductive material, such as, for example, gold, platinum, silver, iridium, carbon, indium tin oxide, indium zinc oxide, copper, aluminum, gallium, iron, mercury amalgams, tantalum, titanium, zirconium, nickel, osmium, rhenium, rhodium, palladium, an organometallic, or a metallic alloy.

Layered on top of base layer 18 and conductive components 20 is a spacer layer 64. Spacer layer 64 may include an electrically insulating material such as polyester. Spacer layer 64 can cover portions of working electrode 22, counter electrode 24, fill-detect anode 28, fill-detect cathode 30, and conductive regions 40-46. In the exemplary embodiment of FIG. 2B spacer layer 64 does not cover electrical contacts 32-38 or auto-on conductor 48. For example, spacer layer 64 can cover a substantial portion of conductive components 20, from a line proximal of contacts 32 and 34 to proximal end 12, except for slot 52 extending from proximal end 12.

A cover 72 may be provided. As shown in FIG. 2B, cover 72 may have a proximal end 74 and a distal end 76 and may be disposed at proximal end 12 of test strip 10 to cover slot 52 thereby partially forming sample chamber 88. Cover 72 can be attached to spacer layer 64 via an adhesive layer 78. Adhesive layer 78 can include a polyacrylic or other adhesive and may include sections disposed on spacer layer 64 on opposite sides of slot 52. A break 84 in adhesive layer 78 extends from distal end 70 of slot 52 to an opening 86. Cover 72 can be disposed on adhesive layer 78 such that proximal end 74 of cover 72 may be aligned with proximal end 12 and distal end 76 of cover 72 may be aligned with opening 86, thereby covering slot 52 and break 84. Cover 72 may be composed of an electrically insulating material, such as polyester. Additionally, cover 72 may be transparent.

Slot 52, together with base layer 18 and cover 72, may define sample chamber 88 in test strip 10, which receives a fluid sample, such as a blood sample, for measurement in the exemplary embodiment. A proximal end 68 of slot 52 can define a first opening in sample chamber 88, through which the fluid sample is introduced. At distal end 70 of slot 52, break 84 can define a second opening in sample chamber 88, for venting sample chamber 88 as a fluid sample enters sample chamber 88. Slot 52 may be dimensioned such that a blood sample applied to its proximal end 68 is drawn into and held in sample chamber 88 by capillary action, with break 84 venting sample chamber 88 through an opening 86, as the fluid sample enters. Moreover, slot 52 may be dimensioned so that the volume of fluid sample that enters sample chamber 88 by capillary action is about 1 micro-liter or less.

Test strip 10 may include one or more reagent layers 90 disposed in sample chamber 88. In the exemplary embodiment, reagent layer 90 contacts a partially exposed portion 54 of working electrode 22. It is also contemplated that reagent layer 90 may or may not contact exposed portion 56 of counter electrode 24. Reagent layer 90 may include chemical components to enable the level of glucose or other analyte in the body fluid, such as a blood sample, to be determined electro-chemically. For example, reagent layer 90 can include an enzyme specific for glucose, such as glucose oxidase or glucose dehydrogenase, and a mediator, such as potassium ferricyanide or ruthenium hexamine. Reagent layer 90 can also include other components, such as buffering materials (e.g., potassium phosphate), polymeric binders (e.g., hydroxypropyl-methyl-cellulose, sodium alginate, microcrystalline cellulose, polyethylene oxide, hydroxyethylcellulose, and/or polyvinyl alcohol), and surfactants (e.g., Triton X-100 or Surfynol 485).

An example of the way in which chemical components of reagent layer 90 may react with glucose in the blood is described next. The glucose oxidase initiates a reaction that oxidizes glucose to gluconic acid and reduces the ferricyanide to ferrocyanide. When an appropriate voltage is applied to working electrode 22, relative to counter electrode 24, the ferrocyanide is oxidized to ferricyanide, thereby generating a current that is related to the glucose concentration in the blood sample.

As depicted in FIG. 2B, the position and dimensions of the layers of test strip 10 may result in test strip 10 having regions of different thicknesses. Of the layers above base layer 18, the thickness of spacer layer 64 may constitute a substantial thickness of test strip 10. Thus the distal end of spacer layer 64 may form a shoulder 92 in test strip 10. Shoulder 92 may delineate a thin section 94 of test strip 10 extending from shoulder 92 to distal end 14, and a thick section 96 of test strip 10 extending from shoulder 92 to proximal end 12. The elements of test strip 10 used to electrically connect it to test meter 200, 208, namely, electrical contacts 32-38 and auto-on conductor 48, can all be located in thin section 94. Accordingly, test meter 200, 208 can be sized and configured to receive thin section 94 but not thick section 96. This may allow the user to insert the correct end of test strip 10, i.e., distal end 14 of thin section 94, and can prevent the user from inserting the wrong end, i.e., proximal end 12 of thick section 96, into test meter 200, 208.

Test strip 10 can be sized for easy handling. For example, test strip 10 may measure approximately 27 mm long (i.e., from proximal end 12 to distal end 14) and about 9 mm wide. According to the exemplary embodiment, base layer 18 may be a polyester material about 0.35 mm thick and spacer layer 64 may be about 0.127 mm thick and cover portions of working electrode 22. Adhesive layer 78 may include a polyacrylic or other adhesive and have a thickness of about 0.013 mm. Cover 72 may be composed of an electrically insulating material, such as polyester, and can have a thickness of about 0.1 mm. Sample chamber 88 can be dimensioned so that the volume of fluid sample held is about 1 micro-liter or less. For example, slot 52 can have a length (i.e., from proximal end 12 to distal end 70) of about 3.6 mm, a width of about 1.52 mm, and a height (which can be substantially defined by the thickness of spacer layer 64) of about 0.10 mm. The dimensions of test strip 10 for suitable use can be readily determined by one of ordinary skill in the art. For example, a meter with automated test strip handling may utilize a test strip smaller than 9 mm wide.

Although FIGS. 2A and 2B show an exemplary embodiment of test strip 10, other configurations, chemical compositions and electrode arrangements could be used. Different arrangements of working electrode 22, counter electrode 24, fill-detect anode 28, and/or fill-detect cathode can also be used. In the configuration shown in FIGS. 2A and 2B, working electrode 22 and counter electrode 24 are separated by boundaries aligned in the x-axis, perpendicular to the length of test strip 10 in the y-axis. Alternatively, working electrode 22 and counter electrode 24 can be separated by boundaries aligned in the y-axis, parallel to the length of test strip 10. It is also contemplated that working electrode 22 and counter electrode 24 may be aligned at any angle relative the length of test strip 10.

### Test Strip Array Configuration

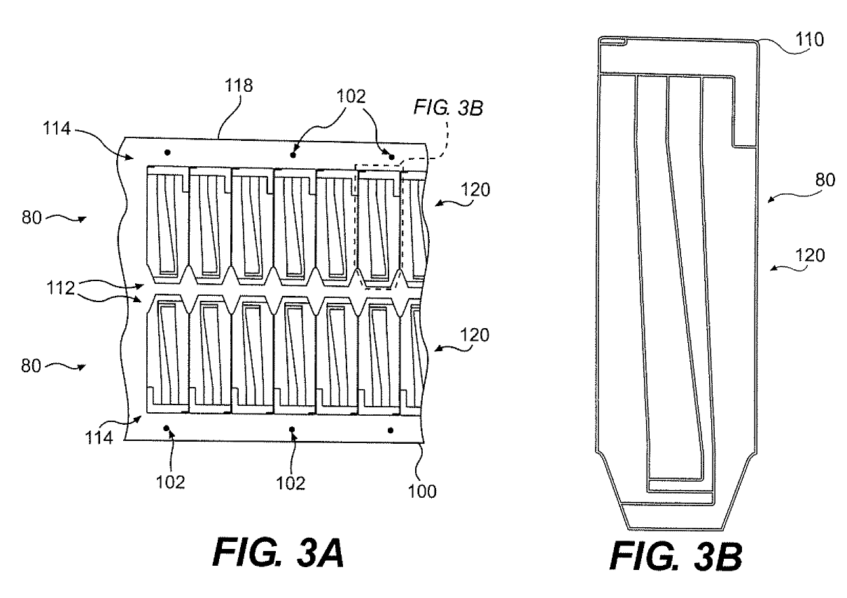

FIG. 3A shows a top view of a reel 100 according to an exemplary disclosed embodiment. The term “reel” as used herein applies to a material of continuous indeterminate length or to sheets of material of determinate length. In some embodiments, reel 100 may include base layer 118. As described below, an array of conductive components 120 may be deposited on base layer 118. Various layers may be added to base layer 118 to form test strip 110 similar to that described in FIG. 2B. Test strips 110 may then be separated from the array of test strips 110 formed on reel 100 to produce multiple individual test strips 110.

A plurality of feature sets 80 may be formed on base layer 118, wherein each feature set 80 may include a plurality of conductive components 120, such as, for example, an electrode, a conductive region and an electrode contact. Feature sets 80 may include any suitable conductive or semi-conductive material. In some embodiments, feature sets 80 can be formed using lift-off lithography or shadow masking, as described in commonly-assigned, copending non-provisional U.S. patent application Ser. No. 11/476,702 “Method of Manufacturing a Diagnostic Test Strip”, filed Jun. 29, 2006, the disclosure of which is hereby incorporated herein by reference in its entirety. It is also contemplated that feature sets 80 may be formed by direct writing, laser ablation, sputtering, screen printing, contact printing or any suitable manufacturing method. One exemplary process is direct writing of electrodes as described in commonly-assigned, copending provisional patent application No. 60/716,120 “Biosensor with Direct Written Electrode”, filed Sep. 13, 2005, the disclosure of which is hereby incorporated herein by reference in its entirety. Another exemplary process is screen printing as described in commonly-assigned, U.S. Pat. No. 6,743,635 “System and methods for blood glucose sensing,” filed Nov. 1, 2002, the disclosure of which is hereby incorporated herein by reference in its entirety.

Following the formation of one or more feature sets 80 on base layer 118, various layers may be added to base layer 118 and feature sets 80 to form a laminate structure as shown in FIG. 2B. Then, individual test strips 110 may be separated from reel 100 via a “singulation” process, wherein the outer shape of test strip 110 formed by the manufacturing process may be represented by the dotted line shown in FIGS. 3A and 3B. In some embodiments, a single feature set 80 may include conductive components 120 of a single test strip 110. Although FIGS. 3A and 3B shows one configuration of feature set 80, it is understood that other configurations of feature set 80 may be used to form test strip 110.

As shown in FIG. 3A, feature sets 80 may be arranged in two rows on reel 100. In the exemplary embodiment depicted, proximal ends 112 of the two rows of feature sets 80 are in juxtaposition in the center of reel 100 and distal ends 114 of feature sets 80 are arranged at the periphery of reel 100. It is also contemplated that proximal ends 112 and distal ends 114 of feature sets 80 can be arranged in the center of reel 100, and distal ends 114 of two rows of feature sets 80 can be arranged in the center of reel 100. Further, the separation distance between feature sets 80 may be designed to permit a single cut to separate adjacent feature sets 80 during a singulation process.

As shown in FIG. 3A, reel 100 includes a plurality of registration points 102 at the distal end 114 of each test strip on reel 100. Registration points 102 may be used during one or more manufacturing processes to locate a feature of test strip 110 relative to reel 100. One or more manufacturing steps may require registration points 102 to ensure precise alignment of laminate layers and/or other manufacturing processes, such as, for example, deposition of conductive components, mask alignment, reagent deposition, singulation, etc. For example, registration points 102 may be used during lamination to ensure that spacer layer 64 is properly positioned over base layer 18 such that slot 52 is positioned to adequately expose portions of electrodes 54, 56, 60, 62 as shown in FIGS. 2A and 2B. Registration points 102 may also be utilized during chemistry deposition, alignment of adjacent layers, singulation, or any other process associated with the formation of test strips 110.

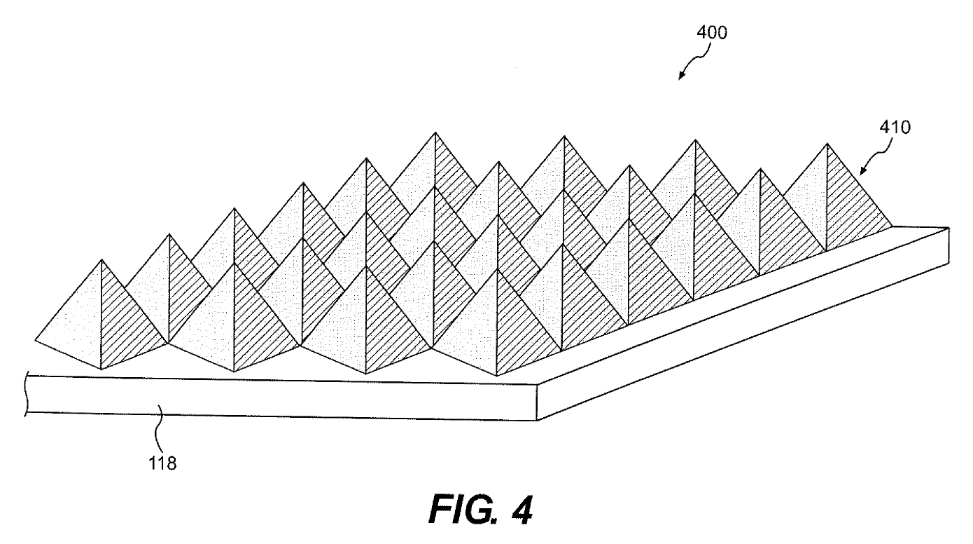

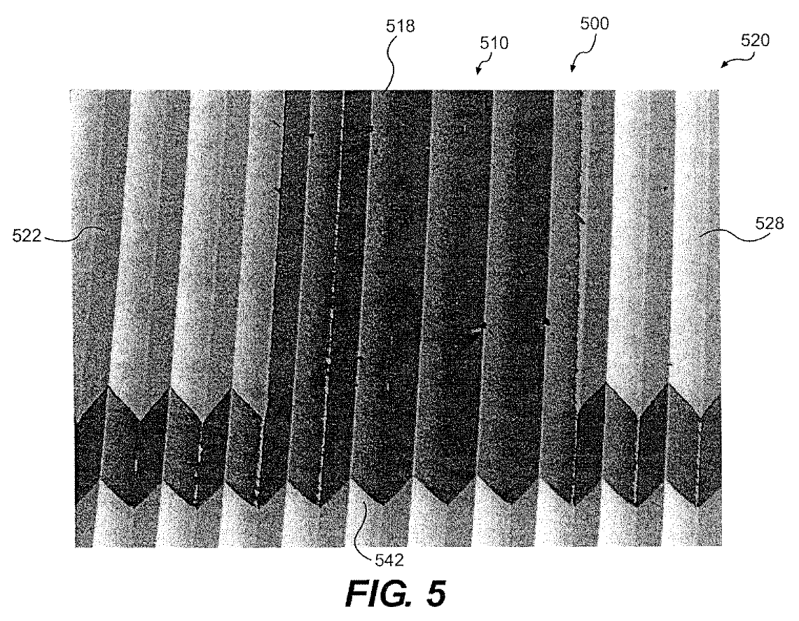

FIG. 4 illustrates a magnified view of base layer 118 including a surface texture 400. Surface texture 400 may include one or more surface features 410 formed on base layer 118 and configured to increase a surface area of base layer 118. For example, surface texture 400 may include one or more pyramidal surface features 410 as shown in FIG. 4. Surface feature 410 may provide an increased surface area relative to a planar surface area of base layer 118 lacking surface feature 410. In some embodiments, surface texture 400 may increase a surface area of base layer 118 by at least 10%, 20%, 50%, 100%, 200%, or 300%.

Increasing a surface area of base layer 118 may offer several advantages over existing planar designs. For example, an increased surface area of base layer 118 will increase an area available for deposition of conductive components 20 and/or reagent layer 90. Increasing an electrode surface area may permit enhanced detection of an electrochemical reaction, such as, for example, as described above for glucose detection. Specifically, conductive components 20 formed on surface texture 400 will have a greater surface area than conductive components 20 formed on a planar base layer 118 without surface texture 400. The increased surface area may enhance signal detection by providing an electrode with a larger surface area to detect a current generated by an electrochemical reaction. Further, by increasing the surface area of one or more conductive components 20, test strip size may be reduced while retaining appropriate signal detection, requiring smaller quantities of materials and/or smaller volumes of body fluid. In addition, an increased surface area may permit improved adhesion between adjacent layers.

Surface texture 400 may include any surface profile or geometry that functions to increases a surface area of a generally planar substrate. For example, surface texture 400 may include one or more protrusions extending from a generally planar substrate. In addition, surface texture 400 may include one or more indentations extending into a generally planar substrate. As shown in FIG. 4, surface texture 400 includes a plurality of pyramidal protrusions, shown as surface feature 410, extending from base layer 118.

In some embodiments, surface texture 400 may include one or more surface features 410 configured to increase an area of a generally planar substrate. Surface feature 410 may include any structure configured to increase the surface area of a generally planar substrate. For example, surface features 410 may include one or more protrusions from base layer 118 and/or indentations into base layer 118 as described above.

Surface features 410 may be any suitable shape or size. For example, surface feature 410 may include a corrugation, a prism, a box-like, a needle, or any other shape that increases a surface area of a generally planar surface. Surface features 410 may also include indentations formed into a surface of a generally planar surface. For example, surface features 410 may include one or more dimples etched into a generally planar surface. In some embodiments, surface texture 400 may include one or more surface features 410 of different and/or similar shape.

Surface features 410 may be any suitable size, such as, for example, approximately 200 micro-meters wide and approximately 100 micro-meters high. In some embodiments, surface features 410 may have a dimension in a range of 100 micrometers to 1 nanometer, wherein the dimension may include a height, width, or depth of surface feature 410. Surface texture 400 may include one or more surface features 410 of different and/or similar size.

In some embodiments, surface features 410 may include smaller structures (not shown) to further increase the surface area of surface features 410. Such smaller structures, termed secondary surface features, may be formed on any suitable location on surface features 410. Surface features 410 may include one or more secondary surface features, wherein the secondary surface features may be of similar and/or different size, shape or spatial distribution. For example, a plurality of secondary surface features may form an array on surface feature 410.

Surface texture 400 may include any suitable spatial distribution of surface features 410 on base layer 118. For example, a plurality of surface features 410 may be arranged to form an array, as shown in FIG. 4. An array may include any pattern of repeated surface features 410. It is also contemplated that surface features 410 may be formed in an irregular arrangement using any suitable process. For example, surface texture 400 may be formed via a deposition process as described below, wherein the deposition of surface features 410 may form an irregular arrangement. Further, surface features 410 may be spatially arranged at any suitable density, such as, for example, a density in a range of 1 surface feature per 10 mm2 to 1,000,000 surface features per mm2. Surface texture 400 may also include surface features 410 at one or more different densities.

In some embodiments, surface texture 400 may be formed in select regions of base layer 118. For example, surface texture 400 may be formed at proximal end 112 of base layer 118 surrounding. It is also contemplated that surface texture 400 may be formed in select regions of test strip 10, such as, for example exposed portion of working electrode 54 and/or exposed portion of counter electrode 56, as shown in FIG. 2A. Surface texture 400 may also cover an entire surface of base layer 118.

Surface texture 400 may be formed on a generally planar substrate using any suitable method. For example, surface features 410 may be formed on base layer 118 using a microreplication process. Such a process may include exposing base layer 118 to heat and/or pressure and then contacting base layer 118 with a suitable tool or die to form surface features 410. Other processes may include thermoforming, traditional embossing, injection molding, plasma etching, and other process to add, transform, or remove material from base layer 118 to form surface texture 400. For example, surface texture 400 may be formed on a substrate by exposure to extreme ultra-violet radiation. It is also contemplated that surface texture 400 may be formed using any suitable type of mold, such as, for example, a rulable mold or a non-rulable mold. Such a mold may be manufactured using any suitable process. For example, a mold may be manufactured as described in U.S. Pat. No. 6,010,609 “Method of making a microprism master mold,” published Jan. 4, 2000, the disclosure of which is hereby incorporated herein by reference in its entirety.

In some embodiments, surface texture 400 may be formed by a deposition process. For example, particles of may be deposited on base layer 118 to more one or more surface features 410. Specifically, surface texture 400 may be formed by sputtering nanoparticles, nanotubes or colloids on any suitably prepared planar substrate. Such a process may form an irregular surface of similar and/or different sized micro- and/or nano-structures.

Surface texture 400 may be formed at any stage during the formation and/or processing of reel 100. For example, reel 100 may be formed with one or more surface features 410 In other embodiments, surface texture 400 may be formed on reel 100 during the test strip manufacturing process. Specifically, surface texture 400 may be formed before, during, or after deposition of conductive components 20 on base layer 118.

### EXAMPLE 1

### Conductive Component Deposition on Surface Texture

FIG. 5 is a scanning electron microscope image of a sample test media showing a magnified view of a junction between three conductive components. A surface texture 500 was formed on a generally planar substrate 518 using a microreplication process. Surface texture 500 includes an array of surface features 510, wherein each surface feature 510 includes a geometry in the form of a corrugation extending from the top to the bottom of the image. As shown, surface features 510 are corrugations with dimensions of approximately 100 microns high and approximately 200 microns wide.

Following formation of surface texture 500, substrate 518 was patterned with a photoresist (not shown). A layer of gold conductive material was then sputtered onto the photoresist patterned substrate. The photoresist was subsequently removed to reveal a plurality of conductive components 520 formed on base layer 518. Conductive components 520 include a working electrode 522, a fill-detect anode 528, and a counter electrode conductive region 542 shown as light shaded regions on surface texture 500. The surface area of conductive components 520 is increased approximately 40% relative to conductive components formed on a generally planar substrate. The gap between working electrode 522 and fill-detect anode 528 is approximately 1000 microns.

### Manufacturing of Test Strips

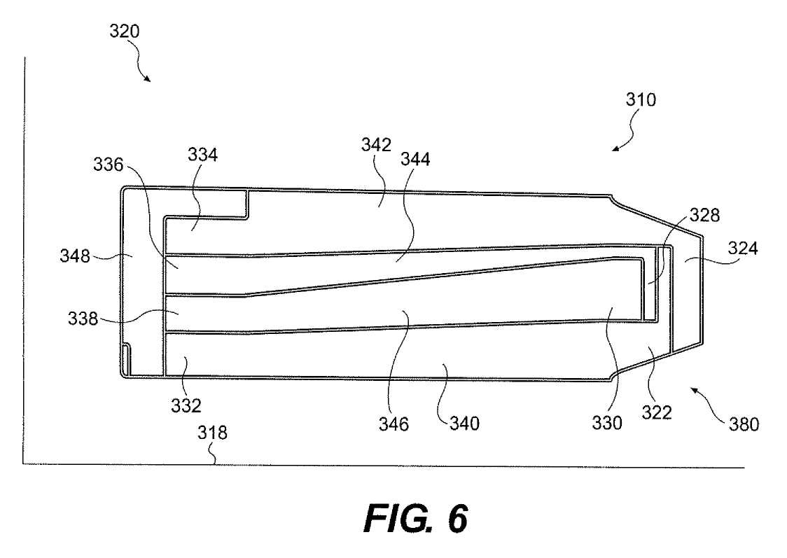

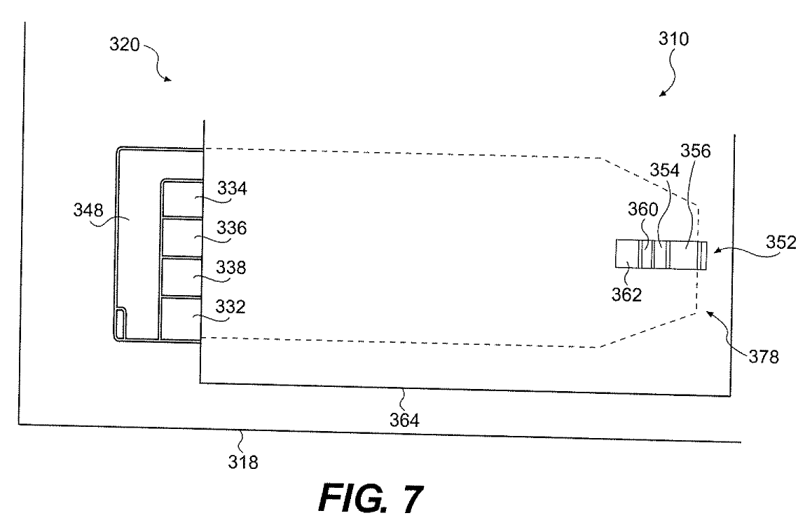

FIGS. 6 and 7 show a partially fabricated test strip structure 310 that may be fabricated as previously described. In each of FIGS. 6 and 7, the outer shape of the test strip 310 that would be formed in the overall manufacturing process is shown as a dotted line in FIG. 7. Although these figures show steps for manufacturing test strip 310 with a configuration similar to that shown in FIGS. 1A, 2A, 2B, it is to be understood that similar steps can be used to manufacture test strips having other configurations of components.

As depicted in the exemplary embodiment shown in FIG. 6, test strip 310 may include a plurality of conductive components 320, such as, for example, electrodes 322, 324, 328 and 330. Conductive components 320 of test strip 310 may be partially formed by forming feature set 380 as discussed above. In some embodiments, conductive components 320 may be at least partially formed by one or more processing techniques. For example, a processing technique, such as laser ablation, may be used to more precisely define the boundaries of some conductive components 320. In other embodiments, a processing technique may include lamination, etching or a physical separation process, such as, for example, stamping and cutting.

Test strip 310 may also include one or more coding regions (not shown), configured to provide coding information on test strip 310. For example, coding regions may include a discrete set of contacting pads as described in commonly-assigned, copending patent application Ser. No. 11/181,778, filed Jul. 15, 2005, entitled “DIAGNOSTIC STRIP CODING SYSTEM AND RELATED METHODS OF USE”, (Attorney Docket 06882-0147), the disclosures of which is hereby incorporated herein by reference in its entirety. The discrete pattern formed by a set of contacting pads may include conducting and non-conducting regions designed to be readable by test meter to identify data particular to the test strip.

Following the formation of feature set 380 on base layer 318, spacer layer 364 can be applied to conductive components 320 and base layer 318, as illustrated in FIG. 7. Spacer layer 364 can be applied to conductive components 320 and base layer 318 in a number of different ways. In an exemplary approach, spacer layer 364 may be provided as a sheet or web large enough and appropriately shaped to cover multiple feature sets 380. In this approach, the underside of spacer layer 364 can be coated with an adhesive to facilitate attachment to conductive components 320 and base layer 318. Various slots can be cut, formed or punched out of spacer layer 364 to shape it before, during or after the application of spacer layer 364 to conductive components 320. For example, as shown in FIG. 7, spacer layer 364 can have a pre-formed slot 352 for each test strip structure. Spacer layer 364 may be positioned over conductive components 320, as shown in FIG. 7, and laminated to conductive components 320 and base layer 318. When spacer layer 364 is appropriately positioned on conductive components 320, exposed electrode portions 354-362 are accessible through slot 352. Similarly, spacer layer 364 leaves contacts 332-338 and auto-on conductor 348 exposed after lamination.

Alternatively, spacer layer 364 could be applied in other ways. For example, spacer layer 64 can be injection molded onto base layer 318 and conductive components 320. Spacer layer 64 could also be built up on base layer 318 and conductive components 320 by screen-printing successive layers of a dielectric material to an appropriate thickness, e.g., about 0.005 inches. An exemplary dielectric material comprises a mixture of silicone and acrylic compounds, such as the “Membrane Switch Composition 5018” available from E.I. DuPont de Nemours & Co., Wilmington, Del. Other materials also could be used, however.

Reagent layer 390 (not shown) can then be applied to each test strip structure after forming spacer layer 364. In an exemplary approach, reagent layer 390 may be applied by dispensing an aqueous composition onto exposed portion 354 of working electrode 322 and letting it dry to form reagent layer 390. It is also contemplated that reagent layer 390 may or may not contact exposed portion 356 of counter electrode 324. An exemplary aqueous composition has a pH of about 7.5 and contains 175 mM ruthenium hexamine, 75 mM potassium phosphate, 0.35% METHOCEL water-soluble cellulose ether, 0.08% TRITON X-100 nonionic surfactant, 5000 u/mL glucose dehydrogenase, 5% sucrose, and 0.05% SILWET L-7608 silicone surfactant. Alternatively, other methods, such as screen-printing, spray deposition, piezo and ink jet printing, can be used to apply the composition used to form reagent layer 390.

Cover 372 (not shown) can then be attached to spacer layer 364, where cover 372 is constructed to cover slot 352, as previously described with respect to FIG. 2B. Portions of the upper surface of spacer layer 364 can also be coated with an adhesive in order to provide adhesive layer 378 to adhere to cover 372. It is also contemplated that cover 372 can include adhesive layer 378 (not shown) configured to adhere to spacer layer 364. Following attachment of cover 372, individual test strips 310 may be separated from the laminated reel. In an exemplary embodiment, the separation process may include stamping or “punching out” individual test strips 310 in a singulation process.

Preferred embodiments of the present invention have been described above. Those skilled in the art will understand, however, that changes and modifications may be made to these embodiments without departing from the true scope and spirit of the invention, which is defined by the claims.

What is claimed is:

1. A biosensor, comprising:
a generally planar substrate including a surface texture configured to increase a surface area of the generally planar substrate; and
at least one conductive component at least partially formed on a portion of the surface texture.

2. The biosensor of claim 1, wherein the surface texture includes a plurality of surface features.

3. The biosensor of claim 2, wherein at least one of the plurality of surface features includes a geometry selected from the group consisting of a corrugation, a pyramid, a box-like structure, a needle, and a dimple.

4. The biosensor of claim 3, wherein the surface texture includes a first surface feature having a first geometry and a second surface feature having a second geometry, wherein the first geometry is substantially similar to the second geometry.

5. The biosensor of claim 3, wherein the surface texture includes a first surface feature having a first geometry and a second surface feature having a second geometry, wherein the first geometry and the second geometry are not the same.

6. The biosensor of claim 2, wherein the plurality of surface features forms an array.

7. The biosensor of claim 2, wherein the plurality of surface features forms an irregular arrangement.

8. The biosensor of claim 2, wherein the plurality of surface features have a density in a range of 1 surface feature per 10 mm² to 1,000,000 surface features per mm².

9. The biosensor of claim 2, wherein at least one of the plurality of surface features has a dimension in a range of 100 micrometers to 1 nanometer.

10. The biosensor of claim 2, wherein at least one of the plurality of surface features further includes a secondary surface feature dimensioned smaller than the at least one of the plurality of surface features.

11. The biosensor of claim 1, wherein the at least one conductive component includes a top surface and a bottom surface, and wherein the top surface has a geometry substantially similar to a geometry of the bottom surface.

12. The biosensor of claim 1, wherein the at least one conductive components includes at least one of an anode, a cathode, a fill-detect electrode, an auto-on conductor, and a coding region.

13. The biosensor of claim 1, wherein the at least one conductive component is formed from at least one of palladium, gold, platinum, silver, iridium, carbon, indium tin oxide, indium zinc oxide, copper, aluminum, gallium, iron, mercury amalgams, tantalum, titanium, zirconium, nickel, osmium, rhenium, rhodium palladium, an organometallic, and a metallic alloy.

14. The biosensor of claim 1, wherein the at least one conductive component is at least partially formed using at least one process selected from the group consisting of sputtering, evaporation, electroplating, ultrasonic spraying, pressure spraying, direct writing, shadow mask lithography, lift-off lithography, and laser ablation.

15. The biosensor of claim 1, wherein the generally planar substrate includes at least one of an acrylic, a PET, and a plastic.

16. The biosensor of claim 1, wherein the surface texture is at least partially formed by a process selected from the group consisting of microreplication, embossing, thermoforming, injection molding, deposition, etching, laser ablation, and extreme ultra-violet radiation.

17. The biosensor of claim 1, wherein the biosensor further includes a reagent layer at least partially deposited on the at least one conductive component.

18. The biosensor of claim 17, wherein the reagent layer is deposited using a process selected from the group consisting of screen-printing, spray deposition, piezo, and ink jet printing.

19. The biosensor of claim 17, wherein the reagent layer includes at least one of glucose oxidase, glucose dehydrogenase, potassium ferricyanide, and ruthenium hexamine.

20. The biosensor of claim 1, wherein the biosensor further includes a dielectric spacer layer at least partially deposited on the at least one conductive component.

21. The biosensor of claim 20, wherein the dielectric spacer layer includes at least one of an acrylic, a PET, and a plastic.

22. The biosensor of claim 20, wherein the biosensor further includes an adhesive layer disposed between the dielectric spacer layer and the at least one conductive component.

23. A method for manufacturing a biosensor, comprising:
forming a surface texture on a generally planar substrate whereby the surface texture increases a surface area of the generally planar substrate; and
at least partially forming at least one conductive component on a portion of the surface texture.

24. The method of claim 23, wherein forming the surface texture includes forming a plurality of surface features.

25. The method of claim 24, wherein forming at least one of the plurality of surface features includes forming at least one surface feature with a geometry selected from the group consisting of a corrugation, a pyramid, a box-like structure, a needle, and a dimple.

26. The method of claim 24, wherein the surface texture includes a first surface feature having a first geometry and a second surface feature having a second geometry, wherein the first geometry is substantially similar to the second geometry.

27. The method of claim 24, wherein the surface texture includes a first surface feature having a first geometry and a second surface feature having a second geometry, wherein the first geometry and the second geometry are not the same.

28. The method of claim 24, wherein forming the surface texture includes forming the plurality of surface features in an array formation.

29. The method of claim 24, wherein forming the surface texture includes forming the plurality of surface features in an irregular arrangement.

30. The method of claim 24, wherein the plurality of surface features are formed with a density in a range of 1 surface feature per 10 mm² to 1,000,000 surface features per mm².

31. The method of claim 24, wherein at least one of the plurality of surface features has a dimension in a range of 100 micrometers to 1 nanometer.

32. The method of claim 24, wherein forming at least one of the plurality of surface features further includes forming a plurality of secondary surface features dimensioned smaller than the at least one of the plurality of surface features.

33. The method of claim 23, wherein at least partially forming the at least one conductive component includes at least partially forming a top surface and at least partially forming a bottom surface, and wherein the top surface has ageometry substantially similar to a geometry of the bottom surface.

34. The method of claim 23, wherein at least partially forming the at least one conductive components includes at least partially forming at least one of an anode, a cathode, a fill-detect electrode, an auto-on conductor, and a coding region.

35. The method of claim 23, wherein the at least one conductive component is formed from at least one of palladium, gold, platinum, silver, iridium, carbon, indium tin oxide, indium zinc oxide, copper, aluminum, gallium, iron, mercury amalgams, tantalum, titanium, zirconium, nickel, osmium, rhenium, rhodium palladium, an organometallic, and a metallic alloy.

36. The method of claim 23, wherein the at least one conductive component is at least partially formed using at least one process selected from the group consisting of sputtering, evaporation, electroplating, ultrasonic spraying, pressure spraying, direct writing, shadow mask lithography, lift-off lithography, and laser ablation.

37. The method of claim 23, wherein the generally planar substrate includes at least one of an acrylic, a PET, and a plastic.

38. The method of claim 23, wherein the surface texture is at least partially formed by a process selected from the group consisting of microreplication, embossing, thermoforming, injection molding, deposition, etching, laser ablation, and extreme ultra-violet radiation.

39. The method of claim 23, wherein the method further includes at least partially depositing a reagent layer on the at least one conductive component.

40. The method of claim 39, wherein the reagent layer is deposited using a process selected from the group consisting of screen-printing, spray deposition, piezo, and ink jet printing.

41. The method of claim 39, wherein the reagent layer includes at least one of glucose oxidase, glucose dehydrogenase, potassium ferricyanide, and ruthenium hexamine.

42. The method of claim 23, wherein the method further includes at least partially depositing a dielectric spacer layer on the at least one conductive component.

43. The method of claim 42, wherein the dielectric spacer layer includes at least one of an acrylic, a PET, and a plastic.

44. The method of claim 42, wherein the method further includes depositing an adhesive layer on the dielectric spacer layer such that the adhesive layer is disposed between the dielectric spacer layer and the at least one conductive component.

45. A reel, comprising:
a generally planar substrate including a surface texture configured to increase a surface area of the generally planar substrate;
at least one conductive component at least partially formed on a portion of the surface texture; and
a plurality of registration points formed on the generally planar substrate.

46. The reel of claim 45, wherein the surface texture includes a plurality of surface features.

47. The reel of claim 46, wherein at least one of the plurality of surface features includes a geometry selected from the group consisting of a corrugation, a pyramid, a box-like structure, a needle, and a dimple.

48. The reel of claim 47, wherein the surface texture includes a first surface feature having a first geometry and a second surface feature having a second geometry, wherein the first geometry is substantially similar to the second geometry.

49. The reel of claim 47, wherein the surface texture includes a first surface feature having a first geometry and a second surface feature having a second geometry, wherein the first geometry and the second geometry are not the same.

50. The reel of claim 46, wherein the plurality of surface features forms an array.

51. The reel of claim 46, wherein the plurality of surface features forms an irregular arrangement.

52. The reel of claim 46, wherein the plurality of surface features have a density in a range of 1 surface feature per 10 mm² to 1,000,000 surface features per mm².

53. The reel of claim 46, wherein at least one of the plurality of surface features has a dimension in a range of 100 micrometers to 1 nanometer.

54. The reel of claim 46, wherein at least one of the plurality of surface features further includes a secondary surface feature dimensioned smaller than the at least one of the plurality of surface features.

55. The reel of claim 45, wherein the at least one conductive component includes a top surface and a bottom surface, and wherein the top surface has a geometry substantially similar to a geometry of the bottom surface.

56. The reel of claim 45, wherein the at least one conductive components includes at least one of an anode, a cathode, a fill-detect electrode, an auto-on conductor, and a coding region.

57. The reel of claim 45, wherein the at least one conductive component is formed from at least one of palladium, gold, platinum, silver, iridium, carbon, indium tin oxide, indium zinc oxide, copper, aluminum, gallium, iron, mercury amalgams, tantalum, titanium, zirconium, nickel, osmium, rhenium, rhodium palladium, an organometallic, and a metallic alloy.

58. The reel of claim 45, wherein the at least one conductive component is at least partially formed using at least one process selected from the group consisting of sputtering, evaporation, electroplating, ultrasonic spraying, pressure spraying, direct writing, shadow mask lithography, lift-off lithography, and laser ablation.

59. The reel of claim 45, wherein the generally planar substrate includes at least one of an acrylic, a PET, and a plastic.

60. The reel of claim 45, wherein the surface texture is at least partially formed by a process selected from the group consisting of microreplication, embossing, thermoforming, injection molding, deposition, etching, laser ablation, and extreme ultra-violet radiation.

61. The reel of claim 45, wherein the biosensor further includes a reagent layer at least partially deposited on the at least one conductive component.

62. The reel of claim 61, wherein the reagent layer is deposited using a process selected from the group consisting of screen-printing, spray deposition, piezo, and ink jet printing.

63. The reel of claim 61, wherein the reagent layer includes at least one of glucose oxidase, glucose dehydrogenase, potassium ferricyanide, and ruthenium hexamine.

64. The reel of claim 45, wherein the biosensor further includes a dielectric spacer layer at least partially deposited on the at least one conductive component.

65. The reel of claim 64, wherein the dielectric spacer layer includes at least one of an acrylic, a PET, and a plastic.

66. The reel of claim 64, wherein the biosensor further includes an adhesive layer disposed between the dielectric spacer layer and the at least one conductive component.

67. The reel of claim 45, wherein the plurality of registration points are formed by a process selected from the group consisting of laser ablation, etching, drilling, printing, punching, scoring, heating, compression and molding.

68. The reel of claim 45, wherein the plurality of registration points are separated by less than 500 mm.

69. The reel of claim 45, wherein at least one of the plurality of registration points is less than 10 mm wide.
